(19) United States
(12) Patent Application Publication
(10) Pub. No.: US 2006/0183758 A1
(43) Pub. Date: Aug. 17, 2006

(54) Method for synthesis of AZA-annelated pyrroles, thiophenes, and furans

(75) Inventors: Charles D. Beard, Montchanin, DE (US); Ving J. Lee, Los Altos, CA (US); C. Ed Whittle, Wilmington, DE (US)

Correspondence Address: SYNNESTVEDT & LECHNER, LLP, 2600 ARAMARK TOWER, 1101 MARKET STREET, PHILADELPHIA, PA, 191072950, US

(73) Assignee: CB Research and Development, Inc., New Castle, DE (US)

(21) Appl. No.: 11/059,744

(22) Filed: Feb. 17, 2005

(51) Int. Cl. A61K 31/519

(52) U.S. Cl. 514/260.1; 544/278; 544/280; 514/265.1

(57) ABSTRACT

Methods of synthesis of intermediates that are useful as bioisosteres of the indole, benzofuran and benzothiophene scaffold are disclosed.

### FIELD OF THE INVENTION

The present invention relates to a novel process for the preparation of functionalized aza-annelated pyrroles, thiophenes, and furans. In particular, the present invention relates to a novel process for the preparation of compounds of formula I which are potential bioisosteres of indole and its derivatives such as, for example, benzofurans and benzothiophenes, in the preparation of therapeutic agents.

### BACKGROUND OF THE INVENTION

Many advances in the life sciences in the 20^{th} century have been due to the discovery of new classes of small molecular weight effectors for various therapeutic needs. The cornerstone of the life sciences is the ability of medicinal chemists to convert primary lead molecules into commercial entities with proper balance of physicochemical properties that enhance in vivo efficacy and retain in vivo activity.

One hallmark of the modern era of medicine has been the decline in morbidity and mortality associated with various acute and chronic conditions. Notably, improvements in drug selectivity and more convenient dosing regimens have been one of the successes of the medicinal chemistry lead optimization process. The collective experience of the past 50 years of drug discovery, in combination with de novo drug design and chemoinformatics data mining methods, have provided medicinal chemists with metrics for drug design. These so-called drug design metrics, often called “drug-likeness” or “drugability”, are primarily based on physicochemical properties of molecules.

These physicochemical properties impact solubility, protein-binding, tissue distribution, first-pass metabolism, excretion, tissue tropism, formulations, and dosing regimen. Depending on the therapeutic indications, the priority parameters that affect a development candidate's efficacy vary. Some indications, as for anti-infective indications, the balance of parameters is very stringent because a drug also has to be taken up into microorganisms. Often these stringent requirements limit structure-activity optimization options for the skilled artisan. Moreover, a lead compound with desired pharmacological activity may have undesirable characteristics that limit its bioavailability or structural features which adversely influence its metabolism and excretion from the body. It may also possess unwanted side effects or toxicity. Thus, it is a major challenge for those skilled in the art to convert a compound binding with high affinity to a biological target (i.e., a “hit” or “lead” molecule) into a successful drug on the market.

Bioisosterism represents one common approach used by those skilled in the art for the rational modification of lead compounds into safer and more clinically effective agents. The terms “bioisostere”, “bioisosteric replacement”, “bioisosterism” and closely related terms as used herein have the same meanings as those generally recognized in the art. For example, a bioisostere is a compound resulting from the exchange of an atom or of a group of atoms with another, broadly similar, atom or group of atoms. The term “bioisostere” can also be used to refer to a portion of an overall molecule, as opposed to the entire molecule itself.

The objective of a bioisosteric replacement in drug development is to create a new compound with similar biological properties to its parent compound by using one bioisostere to replace another with the expectation of maintaining or slightly modifying the biological activity of the first bioisostere. Accordingly, “bioisosterism” arises from a reasonable expectation that a proposed bioisosteric replacement will result in maintenance of similar biological properties. Such a reasonable expectation may be based on structural similarity alone. This is especially true in those cases where a number of particulars are known regarding the characteristic domains of the receptor, etc. involved, to which the bioisosteres are bound or which works upon said bioisosteres in some manner.

Bioisosteres are typically classified as either classical or non-classical. Classical bioisosteres are those that have similar steric and electronic features and have the same number of atoms as the substituent moiety for which they are used as a replacement. Non-classical bioisosteres do not obey the strict steric and electronic definition of classical bioisosteres and they do not have the same numbers of atoms as the substituent moiety for which they are used as a replacement. These bioisosteres are capable of maintaining similar biological activity by mimicking the spatial arrangement, electronic properties, or some other physiochemical property of the molecule or functional group that is critical for the retention of biological activity.

Indole and its derivatives such as, for example, benzofurans and benzothiophenes are well-known to those skilled in the art as desirable building blocks for pharmaceutically-active agents: 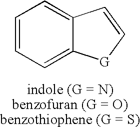
Numerous molecules possessing these structures or their derivatives have been discovered in various biological screening campaigns by life-science companies and universities. For example, U.S. Pat. No. 5,338,849 discloses the use of azaindoles in the treatment of hyperlipidaemia and atherosclerosis; U.S. Pat. No. 5,521,213 discloses the use of diaryl bicyclic heterocycles as inhibitors of cyclooxygenase-2; U.S. Pat. No. 5,714,495 discloses substituted azaindoles, azabenzofurans, and azabenzothiophenes useful for treating a disorder of the melatoninergic system; U.S. Pat. No. 6,025,366 discloses azaindoles derivatives useful as antagonists of GnRH and, hence, potentially useful for the treatment of a variety of sex-hormone related conditions in both men and women; and U.S. Pat. No. 6,169,091 discloses bicyclic heteroaromatic compounds as protein tyrosine kinase inhibitors and their use in medicines in the treatment of psoriasis, fibrosis, atherosclerosis, restenosis, auto-immune disease, allergy, asthma, transplantation rejection, inflammation, thrombosis, nervous system diseases, and cancer. Accordingly, providing promising bioisosteres of such compounds is a major focus in the art of drug development.

Prior art processes for providing such compounds suffer from a number of disadvantages such as, for example, low yields and high complexity. For example, Zhang et al. in J. Org. Chem. 2002, 67, 2345-2347 discloses formation of azaindoles via the Bartoli cyclization, however, the yields are generally very low (20-40%) and the process suffers from little versatility with respect to functionalization of the final azaindole compound. Thibault et al. in Org. Lett., 2003, 5, 26, 5023-5025 discloses formation of azaindoles via N-oxides. This process, however, also suffers from low yields and includes inherent explosion hazards. Katayama et al. in J. Org. Chem. 2001, 66, 3474-3483 disclose formation of indoles, however, here again, the yields are low and the process involves hazardous tin-containing reagents.

Accordingly, there is a need in the art for an improved process for preparing molecules that are potential bioisosteres of, for example, indoles, benzofurans and benzothiophenes.

### SUMMARY OF THE INVENTION

The present invention provides a process for preparing a compound having the structure of Formula (I) 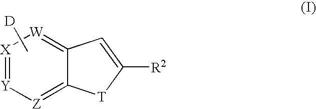
wherein T is NR¹, oxygen, or sulfur, wherein R¹ is hydrogen, substituted or unsubstituted C₁-C₆ alkyl, substituted or unsubstituted C₃-C₇ cycloalkyl, substituted or unsubstituted alkenyl, substituted or unsubstituted alkynyl, substituted or unsubstituted aralkyl, substituted or unsubstituted aryl, or substituted or unsubstituted heteroaryl;

R² is hydrogen, alkyl, haloalkyl, cycloalkyl, (CH₂)ₚOH, (CH₂)_{q}NR¹¹R¹², substituted or unsubstituted aryl, substituted or unsubstituted aralkyl, substituted or unsubstituted heteroaryl, fused substituted or unsubstituted aryl, fused substituted or unsubstituted heteroaryl, or CH(R³)J, wherein

J is hydrogen, alkyl, haloalkyl, CF₃, cycloalkyl, halogen, CHO, CH═NOH, CO₂H, CO₂-alkyl, CN, NO₂, PO(O-alkyl)₂, SO₂-alkyl, S-alkyl, SCF₃, SO₂-aryl, S-aryl, substituted or unsubstituted aryl, substituted or unsubstituted aralkyl, substituted or unsubstituted heteroaryl, fused substituted or unsubstituted aryl, fused substituted or unsubstituted heteroaryl;

R³ is selected from the group of hydrogen, alkyl, haloalkyl, CF₃, cycloalkyl, halogen, CHO, CH═NOH, CO₂H, CO₂-alkyl, CN, NO₂, PO(O-alkyl)₂, SO₂-alkyl, S-alkyl, SCF₃, SO₂-aryl, S-aryl, substituted or unsubstituted aryl, substituted or unsubstituted aralkyl, substituted or unsubstituted heteroaryl, fused substituted or unsubstituted aryl, fused substituted or unsubstituted heteroaryl;

R¹¹ and R¹² are independently hydrogen, alkyl, or alkanoyl; p is 1 to 3; q is 0 to 2; W is CH, CR⁴, or N; X is CH, CR⁵, or N; Y is CH, CR⁶, or N; Z is CH, CR⁷, or N, wherein the total number of nitrogens in W+X+Y+Z is 0-3, and optionally W+X, X+Y, or Y+Z could be joined as either a 5-7 member ring;

R⁴, R⁵, R⁶ and R⁷ are each independently hydrogen, haloalkyl, alkyl, cycloalkyl, (CH₂)ₚOH, halogen, CHO, CH═NOH, CO₂H, CO₂-alkyl, S-alkyl, SO₂-alkyl, S-aryl, (CH₂)_{q}NR¹³R¹⁴, alkoxy, CF₃, SCF₃, NO₂, SO₃H, OH, substituted or unsubstituted aryl, substituted or unsubstituted aralkyl, substituted or unsubstituted heteroaryl, fused substituted or unsubstituted aryl, fused substituted or unsubstituted heteroaryl;

R¹³ and R¹⁴ are independently hydrogen, alkyl, or alkanoyl; and D is H or Br.

The process according to the invention comprises the steps of (a) reacting a compound of the formula 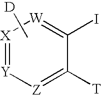
with an acetylene compound selected from the group consisting of 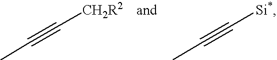
wherein R², D, T, W, X, Y, and Z are as previously defined, I is an iodine atom, and Si* is a silyl-containing acetylene protecting group, and cyclizing the product of step (a) in a protic solvent.

In another aspect, the present invention provides compounds having the structure of Formula (I) 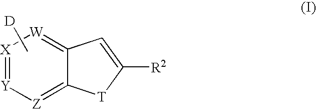
wherein T is NR¹ wherein R¹ is substituted or unsubstituted C₁-C₆ alkyl, substituted or unsubstituted C₃-C₇ cycloalkyl, substituted or unsubstituted alkenyl, substituted or unsubstituted alkynyl, substituted or unsubstituted aralkyl, substituted or unsubstituted aryl, or substituted or unsubstituted heteroaryl;

R² is hydrogen, alkyl, haloalkyl, cycloalkyl, (CH₂)ₚOH, (CH₂)_{q}NR¹¹R¹², substituted or unsubstituted aryl, substituted or unsubstituted aralkyl, substituted or unsubstituted heteroaryl, fused substituted or unsubstituted aryl, fused substituted or unsubstituted heteroaryl, or CH(R³)J, wherein

J is hydrogen, alkyl, haloalkyl, CF₃, cycloalkyl, halogen, CHO, CH═NOH, CO₂H, CO₂-alkyl, CN, NO₂, PO(O-alkyl)₂, SO₂-alkyl, S-alkyl, SCF₃, SO₂-aryl, S-aryl, substituted or unsubstituted aryl, substituted or unsubstituted aralkyl, substituted or unsubstituted heteroaryl, fused substituted or unsubstituted aryl, fused substituted or unsubstituted heteroaryl;

R³ is selected from the group of hydrogen, alkyl, haloalkyl, CF₃, cycloalkyl, halogen, CHO, CH═NOH, CO₂H, CO₂-alkyl, CN, NO₂, PO(O-alkyl)₂, SO₂-alkyl, S-alkyl, SCF₃, SO₂-aryl, S-aryl, substituted or unsubstituted aryl, substituted or unsubstituted aralkyl, substituted or unsubstituted heteroaryl, fused substituted or unsubstituted aryl, fused substituted or unsubstituted heteroaryl;

R¹¹ and R¹² are independently hydrogen, alkyl, or alkanoyl; p is 1 to 3; q is 0 to 2; W is CH, CR⁴, or N; X is CH, CR⁵, or N; Y is CH, CR⁶, or N; Z is CH, CR⁷, or N, wherein the total number of nitrogens in W+X+Y+Z is 0-3, and optionally W+X, X+Y, or Y +Z could be joined as either a 5-7 member ring;

R⁴, R⁵, R⁶ and R⁷ are each independently hydrogen, haloalkyl, alkyl, cycloalkyl, (CH₂)ₚOH, halogen, CHO, CH═NOH, CO₂H, CO₂-alkyl, S-alkyl, SO₂-alkyl, S-aryl, (CH₂)_{q}NR¹³R¹⁴, alkoxy, CF₃, SCF₃, NO₂, SO₃H, OH, substituted or unsubstituted aryl, substituted or unsubstituted aralkyl, substituted or unsubstituted heteroaryl, fused substituted or unsubstituted aryl, fused substituted or unsubstituted heteroaryl; D is H or Br; and R¹³ and R¹⁴ are independently hydrogen, alkyl, or alkanoyl.

In yet another aspect, the present invention provides compoundc having the structure of Formula (I) 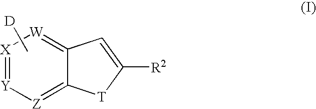
wherein T is selected from NR¹, oxygen, sulfur, wherein

R¹ is hydrogen, substituted or unsubstituted C₁-C₆ alkyl, substituted or unsubstituted C₃-C₇ cycloalkyl, substituted or unsubstituted alkenyl, substituted or unsubstituted alkynyl, substituted or unsubstituted aralkyl, substituted or unsubstituted aryl, or substituted or unsubstituted heteroaryl;

R² is CH(R³)J, wherein J is hydrogen, alkyl, haloalkyl, CF₃, cycloalkyl, halogen, CHO, CH═NOH, CO₂H, CO₂-alkyl, CN, NO₂, PO(O-alkyl)₂, SO₂-alkyl, S-alkyl, SCF₃, SO₂-aryl, S-aryl, substituted or unsubstituted aryl, substituted or unsubstituted aralkyl, substituted or unsubstituted heteroaryl, fused substituted or unsubstituted aryl, fused substituted or unsubstituted heteroaryl;

R³ is selected from the group of hydrogen, alkyl, haloalkyl, CF₃, cycloalkyl, halogen, CHO, CH═NOH, CO₂H, CO₂-alkyl, CN, NO₂, PO(O-alkyl)₂, SO₂-alkyl, S-alkyl, SCF₃, SO₂-aryl, S-aryl, substituted or unsubstituted aryl, substituted or unsubstituted aralkyl, substituted or unsubstituted heteroaryl, fused substituted or unsubstituted aryl, fused substituted or unsubstituted heteroaryl;

p is 1 to 3; q is 0 to 2; W is CH, CR⁴, or N; X is CH, CR⁵, or N; Y is CH, CR⁶, or N; Z is CH, CR⁷, or N, wherein the total number of nitrogens in W+X+Y+Z is 0-3, and optionally W+X, X+Y, or Y+Z could be joined as either a 5-7 member ring;

R⁴, R⁵, R⁶ and R⁷ are each independently hydrogen, haloalkyl, alkyl, cycloalkyl, (CH₂)ₚOH, halogen, CHO, CH═NOH, CO₂H, CO₂-alkyl, S-alkyl, SO₂-alkyl, S-aryl, (CH₂)_{q}NR¹³R¹⁴, alkoxy, CF₃, SCF₃, NO₂, SO₃H, OH, substituted or unsubstituted aryl, substituted or unsubstituted aralkyl, substituted or unsubstituted heteroaryl, fused substituted or unsubstituted aryl, fused substituted or unsubstituted heteroaryl; D is H or Br; and R¹³ and R¹⁴ are independently hydrogen, alkyl, or alkanoyl.

### DETAILED DESCRIPTION OF THE INVENTION

As used herein, the following terms used in the specification and claims have the stated meaning unless otherwise stated:

“Alkyl”, “lower alkyl”, and “C₁-C₆ alkyl” means an aliphatic hydrocarbon group that may be straight or branched having about 1 to about 20 carbon atoms in the chain.

Preferred alkyl groups have 1 to about 12 carbon atoms in the chain. Branched means that one or more lower alkyl groups such as methyl, ethyl or propyl are attached to a linear alkyl chain. “Lower alkyl” means about 1 to about 4 carbon atoms in the chain that may be straight or branched. The alkyl may be substituted with one or more “alkyl group substituents” which may be the same or different, and include halo, cyclo-alkyl, alkoxy, alkoxycarbonyl, aralkyloxycarbonyl, or heteroaralkyloxycarbonyl. Representative alkyl groups include methyl, trifluoromethyl, cyclopropylmethyl, cyclopentylmethyl, ethyl, n-propyl, i-propyl, n-butyl, t-butyl, n-pentyl, 3-pentyl, methoxyethyl, sec-butyl, isopentyl, neopentyl, hexyl, 2-hexyl, 3-hexyl, and 3-methylpentyl.

“Alkynyl” means a straight or branched aliphatic hydrocarbon group of 2 to about 15 carbon atoms that contains at least one carbon--carbon triple bond. Preferred alkynyl groups have 2 to about 12 carbon atoms. More preferred alkynyl groups contain 2 to about 4 carbon atoms. “Lower alkynyl” means alkynyl of 2 to about 4 carbon atoms. The alkynyl group may be substituted by one or more alkyl group substituents as defined herein. Representative alkynyl groups include ethynyl, propynyl, n-butynyl, 2-butynyl, 3-methylbutynyl, n-pentynyl, heptynyl, octynyl, decynyl, and the like.

“Alkanoyl” means straight or branched chain alkanoyl groups having 1-6 carbon atoms, such as, acetyl, propanoyl, butanoyl, pentanoyl, hexanoyl, isobutanoyl, 3-methylbutanoyl, and 4-methylpentanoyl.

“Alkoxy”, “lower alkoxy”, and “C₁-C₆ alkoxy” means straight or branched chain alkoxy groups having 1-6 carbon atoms, such as, for example, methoxy, ethoxy, propoxy, isopropoxy, n-butoxy, sec-butoxy, tert-butoxy, pentoxy, 2-pentyl, isopentoxy, neopentoxy, hexoxy, 2-hexoxy, 3-hexoxy, and 3-methylpentoxy.

“Aralkyl” means an aryl-alkyl-group wherein aryl and alkyl are defined herein. Preferred aralkyls contain a lower alkylene group. Representative aralkyl groups include benzyl, 2-phenethyl, naphthlenemethyl, and the like.

“Aryl” means an aromatic carbocyclic group having a single ring (e.g., phenyl), multiple rings (e.g., biphenyl), or multiple condensed rings in which at least one is aromatic, (e.g., 1,2,3,4-tetrahydronaphthyl, naphthyl, anthryl, or phenanthryl), which is optionally mono-, di-, or trisubstituted with, e.g., halogen, lower alkyl, lower alkoxy, lower alkylthio, trifluoromethyl, lower acyloxy, aryl, heteroaryl, and hydroxy. Preferred aryl groups include phenyl and naphthyl, each of which is optionally substituted as defined herein.

“Cycloalkyl” means a non-aromatic mono- or multicyclic ring system of about 3 to about 10 carbon atoms, preferably about 5 to about 10 carbon atoms. Preferred cycloalkyl rings contain about 5 to about 6 ring atoms. The cycloalkyl is optionally substituted with one or more “ring system substituents” which may be the same or different, and are as defined herein. Representative monocyclic cycloalkyl include cyclopentyl, cyclohexyl, cycloheptyl, and the like. Representative multicyclic cycloalkyl include 1-decalin, norbornyl, adamantyl, and the like. In such cycloalkyl groups and, preferably in the C₅-C₇ cycloalkyl groups, one or two of the carbon atoms forming the ring can optionally be replaced with a hetero atom, such as sulfur, oxygen or nitrogen. Examples of such groups are piperidinyl, piperazinyl, morpholinyl, pyrrolidinyl, imidazolidinyl, oxazolidinyl, perhydroazepinyl, perhydrooxazapinyl, oxepanyl, perhydrooxepanyl, tetrahydrofuranyl, and tetrahydropyranyl. C₃ and C₄ cycloalkyl groups having a member replaced by nitrogen or oxygen include aziridinyl, azetidinyl, oxetanyl, and oxiranyl.

“Halogen” means fluorine, bromine, chlorine, and iodine.

“Heteroaryl” means one or more aromatic ring systems of 5-, 6-, or 7-membered rings containing at least one and up to four heteroatoms selected from nitrogen, oxygen, or sulfur. Such heteroaryl groups include, for example, thienyl, furanyl, thiazolyl, imidazolyl, (is)oxazolyl, pyridyl, pyrimidinyl, (iso)quinolinyl, napthyridinyl, benzimidazolyl, and benzoxazolyl. Preferred heteroaryls are thiazolyl, pyrimidinyl, preferably pyrimidin-2-yl, and pyridyl. Other preferred heteroaryl groups include 1-imidazolyl, 2-thienyl, 1-(or 2-)quinolinyl, 1-(or 2-) isoquinolinyl, 1-(or 2-)tetrahydroisoquinolinyl, and 2-(or 3-)furanyl.

“Heterocyclyl” means a non-aromatic saturated monocyclic or multicyclic ring system of about 3 to about 10 ring atoms, preferably about 5 to about 10 ring atoms, in which one or more of the atoms in the ring system is/are element(s) other than carbon, for example nitrogen, oxygen or sulfur. Preferred heterocyclyls contain about 5 to about 6 ring atoms. The prefix aza, oxa or thia before heterocyclyl means that at least a nitrogen, oxygen or sulfur atom respective-ly is present as a ring atom. The heterocyclyl is optionally substituted by one or more “ring system substituents” which may be the same or different, and are as defined herein. The atom of the heterocyclyl is optionally oxidized to the corresponding N-oxide. Representative mono-cyclic heterocyclyl rings include piperidyl, pyrrolidinyl, piperazinyl, morpholinyl, thiomorpholinyl, thiazolidinyl, 1,3-dioxolanyl, 1,4-dioxanyl, tetrahydrofuranyl, tetrahydrothiophenyl, tetrahydrothiopyranyl, and the like.

The present invention provides a convenient and efficient process for the synthesis of heterocyclic intermediates that are potential bioisosteres to the 10-pi aromatic indoles, including azaindoles, benzofurans and benzothiophenes, while possessing basic sites within the aromatic system. Such compounds include, but are not limited to, the following compounds:

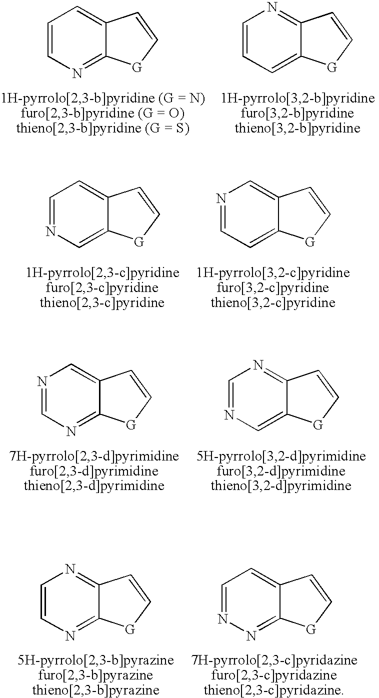

Accordingly, the present invention is directed to a process for the preparation of a compound having the structure of Formula (I) 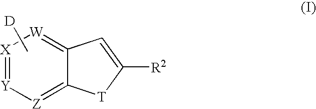
wherein T is NR¹, oxygen, or sulfur, wherein R¹ is hydrogen, substituted or unsubstituted C₁-C₆ alkyl, substituted or unsubstituted C₃-C₇ cycloalkyl, substituted or unsubstituted alkenyl, substituted or unsubstituted alkynyl, substituted or unsubstituted aralkyl, substituted or unsubstituted aryl, or substituted or unsubstituted heteroaryl;

R² is hydrogen, alkyl, haloalkyl, cycloalkyl, (CH₂)ₚOH, (CH₂)_{q}NR¹¹R¹², substituted or unsubstituted aryl, substituted or unsubstituted aralkyl, substituted or unsubstituted heteroaryl, fused substituted or unsubstituted aryl, fused substituted or unsubstituted heteroaryl, or CH(R³)J, wherein

J is hydrogen, alkyl, haloalkyl, CF₃, cycloalkyl, halogen, CHO, CH═NOH, CO₂H, CO₂-alkyl, CN, NO₂, PO(O-alkyl)₂, SO₂-alkyl, S-alkyl, SCF₃, SO₂-aryl, S-aryl, substituted or unsubstituted aryl, substituted or unsubstituted aralkyl, substituted or unsubstituted heteroaryl, fused substituted or unsubstituted aryl, fused substituted or unsubstituted heteroaryl;

R³ is selected from the group of hydrogen, alkyl, haloalkyl, CF₃, cycloalkyl, halogen, CHO, CH═NOH, CO₂H, CO₂-alkyl, CN, NO₂, PO(O-alkyl)₂, SO₂-alkyl, S-alkyl, SCF₃, SO₂-aryl, S-aryl, substituted or unsubstituted aryl, substituted or unsubstituted aralkyl, substituted or unsubstituted heteroaryl, fused substituted or unsubstituted aryl, fused substituted or unsubstituted heteroaryl;

R¹¹ and R¹² are independently hydrogen, alkyl, or alkanoyl; p is 1 to 3; q is 0 to 2; W is CH, CR⁴, or N; X is CH, CR⁵, or N; Y is CH, CR⁶, or N; Z is CH, CR⁷, or N, wherein the total number of nitrogens in W+X+Y+Z is 0-3, and optionally W+X, X+Y, or Y+Z could be joined as either a 5-7 member ring;

R⁴, R⁵, R⁶ and R⁷ are each independently hydrogen, haloalkyl, alkyl, cycloalkyl, (CH₂)ₚOH, halogen, CHO, CH═NOH, CO₂H, CO₂-alkyl, S-alkyl, SO₂-alkyl, S-aryl, (CH₂)_{q}NR¹³R¹⁴, alkoxy, CF₃, SCF₃, NO₂, SO₃H, OH, substituted or unsubstituted aryl, substituted or unsubstituted aralkyl, substituted or unsubstituted heteroaryl, fused substituted or unsubstituted aryl, fused substituted or unsubstituted heteroaryl;

D is H or Br; and

R¹³ and R¹⁴ are independently hydrogen, alkyl, or alkanoyl.

The process according to the invention comprises the steps of (a) reacting a compound of the formula 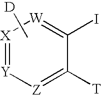
with an acetylene compound selected from the group consisting of 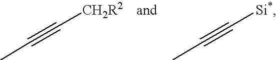
wherein R², D, T, W, X, Y, and Z are as previously defined, I is an iodine atom, and Si* is a silyl-containing acetylene protecting group, and cyclizing the product of step (a) in a protic solvent.

In preferred embodiments of the invention, T is oxygen or NR¹. In a particularly preferred embodiment, R¹ is hydrogen. In another particularly preferred embodiment, R¹ is a substituted or unsubstituted lower alkyl (C₁-C₄). In yet another preferred embodiment, R¹ is a substituted or unsubstituted cycloalkyl (C₃-C₇).

Synthesis

In accordance with the process of the present invention, a unique aspect of the chemistry is the use of regioselective methods for heteroatom-directed functionalization of various azaheterocyclic systems.

In preferred embodiments of the invention, the synthesis of the compounds of the invention is accomplished via either Pathway A or Pathway B.

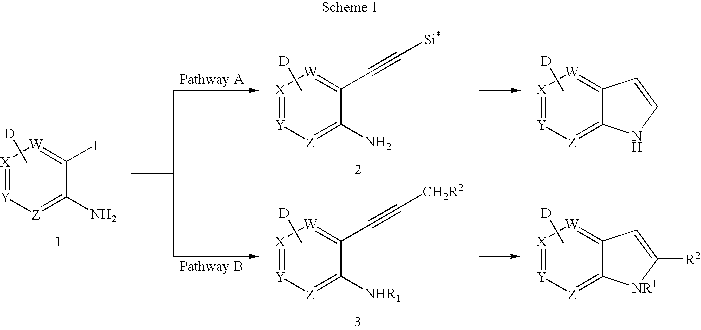

First Reaction—Pathways A and B

According to the present invention, intermediate 1 is readily coupled with the appropriate terminal alkyne under mild conditions in this Sonogashira coupling reaction that has been modified as described herein. A Sonogashira coupling reaction is typically performed in N,N-dimethyl formamide at elevated temperatures so that the solvent undergoes reflux (about 153° C.).

In preferred embodiments, the first reaction illustrated by Scheme 1 generally involves the use of a palladium catalyst either alone or in conjunction with copper iodide. In embodiments where copper iodide is used with the palladium catalyst, the copper reacts with the alkyne to form an alkynylcuprate. Typical palladium catalysts used in such reaction include, for example, trans-PdCl₂(CH₃CN)₂, trans-PdCl₂(PPh₃)₂, Pd(PPh₃)₄, and [Pd(OAc)₂]₃.

In some embodiments of the present invention, the catalyst load can be between about 1 and about 50%. Preferably, the catalyst load is between about 2 and 20%. Most preferably, the catalyst load is about 5% with respect to the amount of intermediate 1.

Applicants have discovered that by varying the solvents from those typically used in a Sonogashira coupling reaction, selective substitution results thus allowing a wide range of sensitive functional group substituents to be tolerated or added at a later time. Applicants have also discovered that by varying the solvents from those typically used in a Sonogashira coupling reaction, the reaction can be successfully performed at lower temperatures.

In preferred embodiments, the solvent is typically selected from a list that includes, for example, dimethylformamide (DMF), N-methylpyrrolidone (NMP), acetonitrile, toluene or tetrahydrofuran. More preferably, toluene or tetrahydrofuran is employed as the solvent. Most preferably, the solvent employed is toluene.

According to one embodiment of the Invention, about 0.025 mol to 5 mol of compound 1 is dissolved in 3.5 L of solvent. Preferably, 0.83 mol to 1.25 mol of compound 1 is dissolved in 3.5 L of solvent. In a particularly preferred embodiment, 1 mol of compound 1 is dissolved in 3.5 L of solvent.

In preferred embodiments, compound 1 is reacted with a molar excess of the appropriate terminal alkyne. Preferably, the molar excess of the terminal alkyne is from 0.9 to 5.0 molar equivalents. More preferably, the molar excess of the terminal alkyne is from 1.0 to 2.0 molar equivalents. Most preferably, the molar excess of the terminal alkyne is about 1.1 molar equivalents.

Any suitable terminal alkyne can be used in the coupling reaction of Reaction 1. Preferably, the terminal alkyne is an acetylene compound selected from the group consisting of 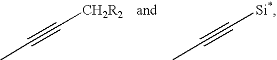
wherein R₂ is defined as above and wherein Si* is a silyl-containing acetylene protecting group such as, for example a trialkylsilyl, alkyldiarylsilyl, or trialkylsilylalkoxy group. Examples of preferred silyl-containing protecting groups for use in accordance with the present invention include trimethylsilyl (TMS), diethylsilyl, tri-isopropylsilyl (TriPS), triethylsilyl, dimethylphenylsilyl, and t-butyl dimethylsilyl (TBDMS). Trimethylsilyl and triethyl silyl groups are more preferred and the trimethylsilyl protecting group is the most preferred.

Preferably, the reaction temperature is maintained between 30° C. and 60° C. and, more preferably, between 40° C. to 45° C. in order to attain the desired selectivity and functional group tolerance. Preferably, the reaction temperature is no greater than 45° C. The reaction mixture is typically subjected to the above conditions for 20 min to 72 hrs. Preferably, the reaction time is between 12 to 16 hrs.

In a preferred embodiment wherein toluene is the solvent employed, the reaction begins at room temperature and heats to between 40° C. to 45° C. as a result of the exothermic reaction that occurs when compound 1, for example, reacts with the acetylene compound in the presence of the catalyst. In this embodiment, an external heat source is not required.

When the reaction is determined to be complete by methods common in the art such as, for example, TLC, HPLC, and GC, the reaction is typically allowed to cool to ambient temperature. Once cooled, the volume of solvent is typically reduced by 40% to 100%. Preferably, the solvent is reduced by 50% for toluene and 100% for tetrahydrofuran. During this stage, the flask is observed for signs of crystallization. If the material (i.e., compound 2 or 3) appears to precipitate, the solid is collected in a Buchner funnel or another device commonly known to those skilled in the art as useful for collecting crystals.

If the material does not precipitate, the reaction product is purified chromatographically on a silica gel column wherein the silica gel is present at about 2 to 10 times by mass of the amount of compound. Preferably, the silica gel is present at about 5 times the amount of compound. The compound is then typically eluted with a mixture of polar solvents and non-polar solvents. Polar solvents that are suitable for use in the present invention include heptanes, pentanes, hexanes, other alkanes, and petroleum ethers. Non-polar solvents suitable for use in the present invention include ethyl acetate, methanol, ethanol, ethers, and low boiling ethers. The preferred combination of polar and non-polar solvents is a 50:50 mixture of ethyl acetate and heptanes.

Second Reaction—Pathways A and B

According to the present invention, the second reaction in pathway A and B forms the bicylcic compound. This reaction is preferably carried out in protic solvents. Protic solvents that are suitable for use in this reaction step include n-butanol, tert-butanol, iso-butanol, iso-propanol, propanol, ethanol, methanol, and mixtures thereof. Tert-butanol is the preferred solvent.

Preferably, an alkoxide base is employeed in the cyclization step. Preferred bases include methoxides, ethoxide, isopropyl oxide, butoxide, tert-butyloxides, etc. used in combination with a lithium, sodium or potassium counter ion. Potassium, sodium or lithium tert-butyloxide is more preferred. Potassium tert-butyloxide is the most preferred.

In preferred embodiments of the invention as described in Pathway A, the reaction temperature for this step is typically maintained between from about 60° C. to 120° C., more preferably between 70° C. and 90° C., and most preferably maintained from about 80° C. to 82° C. in order to attain the desired functional group tolerance. The reaction mixture is subjected to the above conditions for 4 hrs to 48 hrs. Preferably, the reaction time is between 12 to 16 hrs.

In preferred embodiments of the invention as described in Pathway B, the reaction temperature for this step is typically maintained between from about 0° C. to 50° C., more preferably between 20° C. and 40° C., and most preferably from about 20° C. to 25° C. in order to attain the desired functional group tolerance. The reaction mixture is subjected to the above conditions for 10 minutes to 24 hrs. Preferably, the reaction time is between 30 minutes to 2 hrs.

According to one embodiment of the invention as described in both Pathway A and B, about 0.025 mol to 2 mol of compound 2 or 3 (depending on which Pathway) is dissolved in 3.5 L of solvent. Preferably, 0.83 mol to 1.25 mol is dissolved in 3.5 L of solvent. More preferably, 1 mol of compound is dissolved in 3.5 L of solvent.

When the reaction is determined to be complete by methods well known to those skilled in the art such as, for example, TLC, HPLC, and GC, the reaction is typically allowed to cool to ambient temperature. If the reaction will not go to completion an inorganic acid may be added to drive the reaction to completion. Suitable inorganic acids include hydrochloric acid, hydrobromic acid, hydroiodic acid, sulfuric acid, and nitric acid. The preferred acid is hydrochloric acid. The inorganic acid can be added in various concentrations such as, for example, from less than 1 M to concentrated inorganic acid. Preferably, concentrated inorganic acid is employed.

If acid is necessary to drive the reaction to completion, the reaction mixture is preferably heated at a temperature of between 60° C. and 120° C., more preferably between 80° C. and 85° C. for at least 5 minutes or as long as 24 hours. Preferably the reaction is heated for from about 1 hr to 3 hrs and, more preferably, for 1 hr.

Subsequently, the reaction is cooled to ambient temperature and the pH is adjusted to a range of 6 to 8 pH units with solid or aqueous compositions comprising an inorganic base such as, for example, sodium hydroxide, potassium hydroxide, calcium hydroxide, potassium carbonate, sodium carbonate, calcium carbonate, sodium bicarbonate, potassium bicarbonate, and mixtures thereof. Preferably, the pH is adjusted to 7. The inorganic base can be added in various concentrations such as, for example, from less than 1 M to concentrated inorganic base. Preferably, concentrated inorganic base is employed.

Once neutralized, the reaction solvent is typically removed under reduced pressure and the resulting aqueous slurry is extracted with an aprotic solvent. Preferred aprotic solvents for this extraction include ethyl acetate, dichloromethane, toluene, and diethyl ether. Ethyl acetate is the preferred aprotic solvent. Preferably, the extraction is carried out three times with a volume of aprotic solvent that is equal to that of the water layer.

When the volume is reduced by half and cooled to preferably from 0° C. to 30° C., more preferably to 20° C., the compound will typically precipitate out of the solvent. If precipitation is observed, the solid can be collected in, for example, a Buchner funnel and washed again with a cooled volume of the aprotic solvent.

If the material does not precipitate, the reaction product is purified chromatographically on a silica gel column wherein the silica gel is present at about 2 to 10 times the amount of compound. Preferably, the silica gel is present at about 5 times the amount of compound. The compound is eluted with a mixture of two polar solvents (ethyl acetate, methanol, ethanol, ethers, low boiling ethers) and one non-polar solvent (heptanes, pentanes, hexanes, other alkanes, petroleum ethers). The ideal combination is that of ethanol (10%), ethyl acetate (45%), and heptanes (45%).

Compounds of the present invention may exist in different stereoisomeric forms. These compounds can be, for example, racemates or optically active forms. In these situations, the single enantiomers, i.e., optically active forms, can be obtained by asymmetric synthesis or by resolution of the racemates. Resolution of the racemates can be accomplished, for example, by conventional methods such as crystallization in the presence of a resolving agent, or chromatography, using, for example a chiral HPLC column.

Compounds of the present invention will have certain physicochemical properties that can enhance the drug-like characteristics of experimental agents. Such properties include, but are not limited to oral bioavailability, low toxicity, low serum protein binding and desirable in vitro and in vivo half-lives. Assays may be used to predict these desirable pharmacological properties.

Assays used to predict bioavailability include transport across human intestinal cell monolayers, including Caco-2 cell monolayers. Serum protein binding may be predicted from albumin binding assays. Such assays are described in a review by Oravcová et al. (1996, J. Chromat B 677: 1-27). Compound half-life is inversely proportional to the frequency of dosage of a compound. In vitro half-lives of compounds may be predicted from assays of microsomal half-life as described by Kuhnz and Gieschen (Drug Metabolism and Disposition, (1998) volume 26, pages 1120-1127).

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and is typically expressed as the ratio between LD₅₀ and ED₅₀. Compounds that exhibit high therapeutic indices are preferred.

The data obtained from these cell culture assays and animal studies can be used in formulating a range of dosage for use in humans. The dosage of such compounds lies preferably within a range of circulating concentrations that include the ED₅₀ with little or no toxicity. The dosage can vary within this range depending upon the dosage form employed and the route of administration utilized. The exact formulation, route of administration and dosage can be chosen by the individual physician in view of the patient's condition. (See, e.g. Fingl et al., 1975, in “The Pharmacological Basis of Therapeutics”, Ch. 1, p. 1).

The disclosures in this application of all articles and references, including patents, are incorporated herein by reference.

In carrying out the procedures of the present invention it is to be understood by those of ordinary skill in the art that reference to particular synthetic procedures and reagents are not intended to be limiting, but are to be read so as to include all related materials that one of ordinary skill in the art would recognize as being of interest or value in the particular context in which that discussion is presented. For example, it is often possible to substitute one organometallic reagent or reaction condition for another and still achieve similar, if not identical, results. Those skilled in the art will have sufficient knowledge of such systems and methodologies so as to be able, without undue experimentation, to make such substitutions as will optimally serve their purposes in using the methods and procedures disclosed herein.

The invention is described in more detail in the following non-limiting examples. It is to be understood that these methods and examples in no way limit the invention to the embodiments described herein and that other embodiments and uses will no doubt suggest themselves to those skilled in the art.

The following examples present typical syntheses as described in Reaction Scheme 1. These examples are understood to be illustrative only and are not intended to limit the scope of the present invention in any way. As used herein, the following terms have the indicated meanings: “g” refers to grams; “mmol” refers to millimols; “mL” refers to milliliters; “bp” refers to boiling point; “mp” refers to melting point; “° C.” refers to degrees Celsius; “mm Hg” refers to millimeters of mercury; “μL” refers to microliters; “μg” refers to micrograms; and “μM” refers to micromolar.

### EXAMPLES

Proton NMR are recorded on Varian AS 400 spectrometer and chemical shifts are reported as δ (ppm) down field from tetramethylsilane. Mass spectra are determined on Micromass Quattro II.

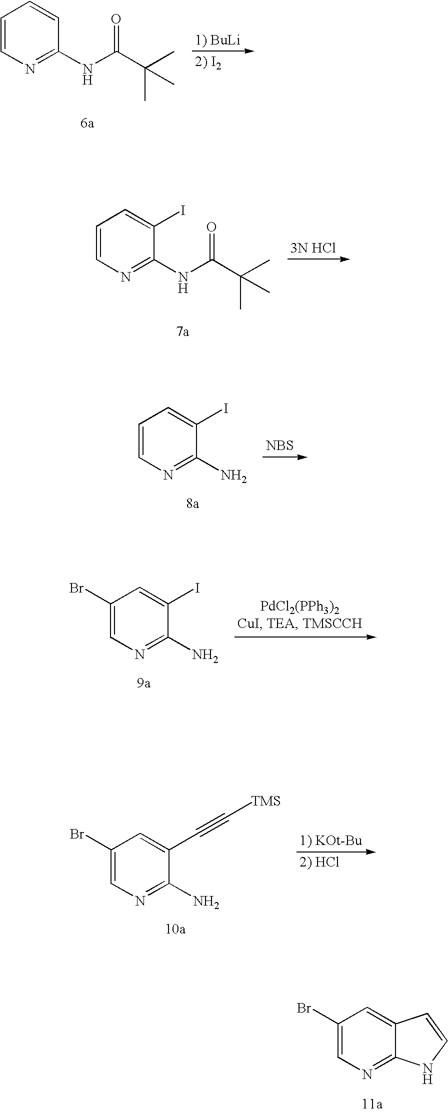

3-Iodo-2-(pivaloylamino)pyridine (7a). A cold solution (−78° C.) of 2-pivaloylamino-pyridine (6a, 500 g, 2.8 mol) in tetrahydrofuran (6 L) was treated with n-butyllithium (2.5 M in hexanes, 2.25 L, 5.63 mol) at a rate such that the temperature did not exceed −55° C. The mixture was stirred for 1 hour until metallation was determined to be complete. A solution of iodine (782 g, 3.01 mol) in tetrahydrofuran (1 L) was added at a rate that the temperature did not exceed −65° C. Upon complete addition, the reaction was stirred for 2 hours and the reaction mixture was slowly poured into ice water (6 L). The mixture was diluted with ethyl acetate (6 L) and the layers separated. The aqueous was washed with ethyl acetate (4 L) and then the combined organics washed with a solution of sodium thiosulfate in water (200 g per liter, 3×4 L) followed by washing with aqueous saturated sodium chloride solution (2×4 L). The organics were dried over sodium sulfate and solvent removed under reduced pressure to yield 7a as a tan to brown solid of sufficient purity to take into the next step. Yield: 800 g, 94%.

3-Iodo-2-aminopyridine (8a). In a flask fitted with a mechanical stirrer, a Dean Stark trap and condenser was added 7a (800 g, 2.63 mol) and 3N hydrochloric acid (8 L). The solution was heated at reflux for 10 hours as the pivalic acid was distilled off. The reaction was cooled to ambient temperature and neutralized by the addition of 50% sodium hydroxide to a pH of 8. The mixture was extracted with ethyl acetate (2×8 L), the organics dried over sodium sulfate and the solvent removed under reduced pressure. The residue was purified by Kugelrohr distillation to give 8a as an off-white solid. Yield: 520 g, 90%.

5-Bromo-3-iodopyridine-2-amine (9a). A flask fitted with a mechanical stirrer, a nitrogen inlet and a thermocouple was placed into a water bath and charged with 8a (520 g, 2.37 mol) and dichloromethane (10 L). To the solution was added solid N-bromosuccinimide (373 g, 2.133 mol) in portions over a 30 minute period. The reaction was stirred for 2 hours and the progress was checked by HPLC. More N-bromosuccinimide was added in small portions until complete consumption of starting material as determined by HPLC (˜40 g). The solution was poured into warm water (6 L), layers separated and the organics washed with warm water (2×6 L), an aqueous solution of sodium thiosulfate (200 g per liter, 2×4 L), followed by washing with saturated sodium chloride solution (6 L). The organics were dried over sodium sulfate and solvent removed under reduced pressure. The residue was purified by passing through a short bed of silica to yield 9a as a tan solid. Yield: 620 g, 88%.

5-Bromo-3-(2-(trimethylsilyl)ethynyl)pyridin-2-amine (10a). To a flask fitted with a mechanical stirrer, a nitrogen inlet, and a thermowell was added 9a (620 g, 2.08 mol) in toluene (6 L). This flask was sparged with nitrogen for 20 minutes and then dichloropalladium bis(triphenyphosphine) (117 g, 0.17 mol) and copper iodide (32 g, 0.17 mol) was added. The solution was sparged with nitrogen for additional 20 minutes and triethyl-amine (318 mL, 2.29 mol) was added followed by quick addition of trimethylsilylacetylene (323 mL, 2.29 mol). The reaction exotherms up to 50° C. over the course of 30 minutes. The reaction is stirred for 12 hours and then poured into water (4 L). The layers were separated and the organics washed with water (4 L) followed by brine (4 L), dried over sodium sulfate and solvent removed under reduced pressure. The residue was passed through a short bed of silica (10% ethyl acetate in heptane) and the fractions containing product were concentrated in vacuo and the residue triturated with pentane, filtered and dried to give 10a as a tan solid. Yield: 448 g, 80%.

5-Bromo-1H-pyrrolo[2,3-b]pyridine (11a). A solution of 10a (440 g, 1.63 mol) in t-butanol (5 L) was treated with potassium t-butoxide (732 g, 6.54 mol). The reaction was heated at reflux for 12 hours. Progress of the reaction was monitored by HPLC. First, quick trimethylsilyl deprotection occurs, over time the t-butyl enol is noted in the HPLC. Once the HPLC indicates that all material is converted to the enol, the reaction is cooled to room temperature and concentrated hydrochloric acid (1 L) is added. The reaction is again brought to reflux and stirred for 12 hours. Once the reaction is determined to be complete by HPLC, the reaction is cooled to room temperature, poured into water (5 L) and filtered through a bed of Celite (diatomaceous earth). The aqueous solution is then diluted with water (5 L) and made basic by the addition of 50% sodium hydroxide. The mixture is extracted with ethyl acetate (3×6 L). The organics are washed with water (4 L) and saturated sodium chloride (4 L), dried over sodium sulfate and solvent removed under reduced pressure to a thick slurry. The slurry is filtered and solids washed with 50% methyl t-butyl ether/heptane (500 mL). The pale yellow solid 11a are dried in a vacuum oven to constant weight, 192 g (60%); ¹H NMR δ (300 MHz, CDCl₃): δ 6.47 (m, 1 H); 7.38 (m, 1 H): 8.08 (d, 1 H); 8.37 (s. 1 H); 10.85 (bs. 1 H). 13 C NMR (300 MHz, CDCl3): δ 100.76. 111.50. 122.31, 126.88. 131.33. 143.26, 147.17.

TABLE 1
Azaindoles via Pathway A

| | |
|---|---|
| | 11 |
| 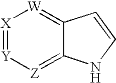 | |

| | W | X | Y | Z |
|---|---|---|---|---|
| 11a | CH | CBr | CH | N |
| 11b | CH | CF | CH | N |
| 11c | CH | CF | CF | N |
| 11d | CF | CF | CF | N |
| 11e | C—Me | CF | CF | N |
| 11f | CH | C—CN | C—CN | N |
| 11g | CH | C—CO₂Me | C—CO₂Me | N |
| 11h | C—CF₃ | C—Br | C—Me | N |
| 11i | N | CH | CH | CH |
| 11j | N | C—Me | CH | CH |
| 11k | N | C—Me | C—Me | N |
| 11m | N | C—CO₂Me | C—Me | N |
| 11n | CH | N | CH | N |
| 11o | CH | N | C—Me | N |
| 11p | C—Me | N | C—Me | N |
| 11q | CH | CH | N | N |
| 11r | C—Me | C—SMe | N | N |
| 11s | N | CH | CH₂CO₂Me | CH |
| 11t | N | CH | C—CH₂F | C—Me |
| 11u | CH | C—CH₂NMe₂ | CH | N |
| 11v | CH | CH | C—CH₂NMe₂ | N |
| 11w | C—c-C₃H₅ | N | C—NMe₂ | N |
| 11x | C—OMe | N | C—c-C₃H₅ | N |
| 11y | N | C—Me | N | C—CH₂OMe |
| 11z | CH | CH | C—OMe | N |
| 11aa | CH | CH | C—OH | N |

### 5-Fluoro-1H-pyrrolo[2,3-b]pyridine (11b)

This was similarly prepared as for 11a, except 5-fluoro-3-(2-(trimethylsilyl)-ethynyl)pyridin-2-amine (10b) was used. Intermediate 10b was prepared from 5-fluoro-3-iodopyridin-2-amine (9b).

### 5,6-Difluoro-1H-pyrrolo[2,3-b]pyridine (11c)

This was similarly prepared as for 11a, except 5,6-difluoro-3-(2-trimethyl-silyl)ethynyl)pyridin-2-amine (10c) was used. Intermediate 5c was prepared from 5,6-difluoro-3-iodopyridin-2-amine (9c).

### 4,5,6-Trifluoro-1H-pyrrolo[2,3-b]pyridine (11d)

This was similarly prepared as for 11a, except 4,5,6-trifluoro-3-(2-trimethyl-silanyl)ethynyl)pyridin-2-amine (10d) was used. Intermediate 10d was prepared from 4,5,6-trifluoro-3-iodopyridin-2-amine (9d).

### 5,6-Difluoro-4-methyl-1H-pyrrolo[2,3-b]pyridine (11e)

This was similarly prepared as for 11a, except 5,6-difluoro-4-methyl-3-(2-trimethylsilyl)ethynyl)pyridin-2-amine (10e) was used. Intermediate 10e was prepared from 5,6-difluoro-3-iodo-4-methylpyridin-2-amine (9e).

### 1H-Pyrrolo[2,3-b]pyridine-5,6-dicarbonitrile (11f)

This was similarly prepared as for 11a, except 6-amino-5-(2-trimethylsilyl)-ethynyl)pyridine-2,3-dicarbonitrile (10f) was used. Intermediate 10f was prepared from 6-amino-5-iodopyridine-2,3-dicarbonitrile (9f).

### Dimethyl 1H-pyrrolo[2,3-b]pyridine-5,6-dicarboxylate (11g)

This was similarly prepared as for 11a, except dimethyl 6-amino-5-(2-(trimethyl-silyl)ethynyl)pyridine-2,3-dicarboxylate (10g) was used. Intermediate log was prepared from dimethyl 6-amino-5-iodopyridine-2,3-dicarboxylate (9g).

### 5-Bromo-4-(trifluoromethyl)-6-methyl-1H-pyrrolo[2,3-b]pyridine (11h)

This was similarly prepared as for 11a, except 5-bromo-4-(trifluoromethyl)-6-methyl-3-(2-(trimethylsilyl)ethynyl)pyridine-2-amine (10h) was used. Intermediate 5h was prepared from 5-bromo-4-(trifluoromethyl)-3-iodo-6-methylpyridin-2-amine (9h).

### 1H-Pyrrolo[3,2-b]pyridine (11i)

This was similarly prepared as for 11a, except 2-(2-(trimethylsilyl)ethynyl)-pyridine-3-amine (10i) was used. Intermediate 10i was prepared from 2-iodopyridin-3-amine (9i).

### 5-Methyl-1H-pyrrolo[3,2-b]pyridine (11j)

This was similarly prepared as for 11a, except 6-methyl-2-(2-(trimethylsilyl)-ethynyl)pyridine-3-amine (10j) was used. Intermediate 10j was prepared from 2-iodo-6-methylpyridin-3-amine (9j).

### 2,3-Dimethyl-5H-pyrrolo[2,3-b]pyrazine (11k)

This was similarly prepared as for 11a, except 5,6-dimethyl-3-(2-(t-butyl-dimethylsilyl)ethynyl)pyrazin-2-amine (10k) was used. Intermediate 10k was prepared from 3-iodo-5,6-dimethylpyrazin-2-amine (9k).

### Methyl 3-Methyl-5H-pyrrolo[2,3-b]pyrazine-2-carboxylate (11m)

This was similarly prepared as for 11a, except methyl 5-amino-3-methyl-6-(2-(t-butyldimethylsilyl)ethynyl)pyrazin-2-carboxylate (10m) was used. Intermediate 10m was prepared from methyl 5-amino-6-iodo-3-methylpyrazine-2-carboxylate (9m).

### 7H-Pyrrolo[2,3-c]pyrimidine (6n)

This was similarly prepared as for 6a, except 5-(2-(trimethylsilyl)ethynyl)-pyrimidin-4-amine (5n) was used. Intermediate 5n was prepared from 5-iodo-pyrimidin-4-amine (4n).

### 2-Methyl-7H-Pyrrolo[2,3-c]pyrimidine (11o)

This was similarly prepared as for 11a, except 2-methyl-5-(2-(trimethylsilyl)-ethynyl)pyrimidin-4-amine (100) was used. Intermediate 100 was prepared from 5-iodo-2-methylpyrimidin-4-amine (9o).

### 2,4-Dimethyl-7H-pyrrolo[2,3-c]pyrimidine (11p)

This was similarly prepared as for 11a, except 2,6-dimethyl-5-(2-(trimethyl-silyl)ethynyl)pyrimidin-4-amine (10p) was used. Intermediate 10p was prepared from 5-iodo-2,6-dimethylpyrimidin-4-amine (9p).

### 7H-Pyrrolo[2,3-c]pyridazine (11q)

This was similarly prepared as for 11a, except 4-(2-(trimethylsilyl)ethynyl)-pyridazin-3-amine (10q) was used. Intermediate 10q was prepared from 4-iodo-pyridazin-3-amine (9q).

### 4-Methyl-3-(methylthio)-7H-pyrrolo[2,3-c]pyridazine (11r)

This was similarly prepared as for 11a, except 5-methyl-4-(2-(trimethylsilyl)-ethynyl)-6-(methylthio)pyridazin-3-amine (10r) was used. Intermediate 10r was prepared from 4-iodo-5-methyl-6-(methylthio)pyridazin-3-amine (9r).

### Methyl 2-(1H-pyrrolo[2,3-c]pyridine-6-yl)acetate (11s)

This was similarly prepared as for 11a, except methyl 2-(5-amino-6-(2-(trimethyl-silyl)ethynyl)pyridine-3-yl)acetate (10s) was used. Intermediate 10s was prepared from methyl 2-(5-amino-6-iodopyridin-3-yl)acetate (9s).

### 6-(Fluoromethyl)-7-methyl-1H-pyrrolo[3,2-b]pyridine (11t)

This was similarly prepared as for 11a, except 5-(fluoromethyl)-4-methyl-2-(2-(trimethylsilyl)ethynyl)pyridin-3-amine (10t) was used. Intermediate 10t was prepared from 5-(fluoro-methyl)-2-iodo-4-methylpyridin-3-amine (9t).

### N,N-Dimethyl(1H-pyrrolo[2,3-b]pyridine-5-yl)methanamine (11u)

This was similarly prepared as for 11a, except 5-((dimethylamino)methyl)-3-(2-(trimethylsilyl)ethynyl)pyridin-2-amine (10u) was used. Intermediate 10u was prepared from 5-((dimethylamino)methyl)-3-iodopyridin-2-amine (9u).

### N,N-Dimethyl(1H-pyrrolo[2,3-b]pyridine-6-yl)methanamine (11v)

This was similarly prepared as for 11a, except 6-((dimethylamino)methyl)-3-(2-(trimethylsilyl)ethynyl)pyridin-2-amine (10v) was used. Intermediate 10v was prepared from 6-((dimethylamino)methyl)-3-iodopyridin-2-amine (9v).

### 4-Cyclopropyl-N,N-dimethyl-7H-pyrrolo[2,3-d]pyrimidin-2-amine (11w)

This was similarly prepared as for 11a, except 6-cyclopropyl-N²,N²-dimethyl-5-(2-(trimethylsilyl)ethynyl)pyrimidine-2,4-diamine (10w) was used. Intermediate 10w was prepared from 6-cyclopropyl-5-iodo-N², N²-dimethylpyrimidin-2,4-diamine (9w).

### 2-Cyclopropyl-4-methoxy-7H-pyrrolo[2,3-d]pyrimidine (11x)

This was similarly prepared as for 11a, except 2-cyclopropyl-6-methoxy-5-(2-(trimethylsilyl)ethynyl)pyrimidine-4-amine (10x) was used. Intermediate 10x was prepared from 2-cyclopropyl-5-iodo-6-methoxypyrimidin-4-amine (9x).

### 4-(Methoxymethyl)-2-methyl-5H-pyrrolo[3,2-d]pyrimidine (11y)

This was similarly prepared as for 11a, except 6-(methoxymethyl)-2-methyl-6-(2-(trimethylsilyl)ethynyl)pyrimidine-5-amine (10y) was used. Intermediate 10y was prepared from 4-iodo-6-(methoxymethyl)-2-methylpyrimidin-5-amine (9y).

### 6-Methoxy-1H-pyrrolo[2,3-b]pyridine (11z)

This was similarly prepared as for 11a, except 6-methoxy-3-(2-(trimethylsilyl)-ethynyl)pyridin-2-amine (10z) was used. Intermediate 10z was prepared from 3-iodo-6-methoxypyridin-2-amine (9z).

### 1H-Pyrrolo[2,3-b]pyridine-6-ol (11aa)

### [1H-Pyrrolo[2,3-b]pyridin-6(7H)-one (keto tautomer)]

This was similarly prepared as for 11a, except 6-hydroxy-3-(2-(trimethylsilyl)-ethynyl)pyridin-2-amine (10aa) was used. Intermediate 10aa was prepared from 3-iodo-6-hydroxypyridin-2-amine (9aa).

### 2-Benzyl-1H-pyrrolo[2,3-b]pyridine (13a)

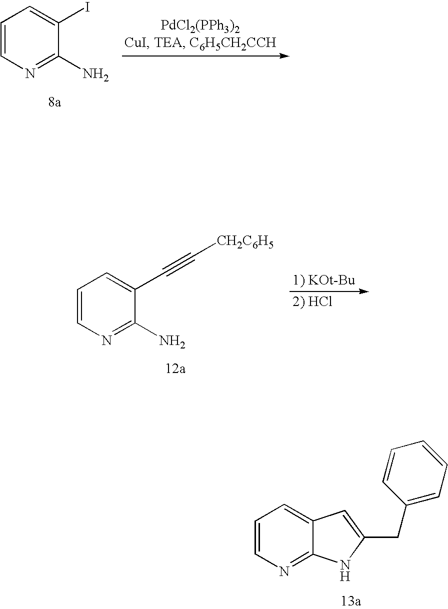

3-(3-Phenylprop-1-ynyl)pyridine-2-amine (12a). To a flask fitted with a nitrogen inlet and a thermowell was added toluene (100 mL) and 2-amino-3-iodopyridine (8a, 10 g, 45.5 mmol). The oxygen was removed from solution by passing a steady stream of nitrogen through the solution for 20 minutes. The dichloropalladium bis-triphenylphosphine (1.9 g, 2.7 mmol) and copper iodide (0.5 g, 2.7 mmol) was added to the reaction flask, followed by the addition of triethyl amine (6.9 mL, 50 mmol) and benzylacetylene (5.8 g, 50 mmol) causing an exotherm to 40° C. over the course of 30 minutes. The resulting slurry was stirred overnight at ambient temperature, poured into water (300 mL) and diluted with ethyl acetate (200 mL). The layers were separated and the organics washed with brine, dried over sodium sulfate, filtered and the solvent removed under reduced pressure. The residue was purified by passing through a short column of silica gel (100 g) eluting with 20% ethyl acetate in heptanes to give the product as a yellow solid. Yield: 6.2 g, 65%.

2-Benzyl-1H-pyrrolo[2,3-b]pyridine (13a). In a flask fitted with a reflux condenser, nitrogen inlet and thermowell was added 12a (6 g, 28.8 mmol) and t-butanol (100 mL). To this mixture was added potassium t-butoxide (16 g, 144 mmol) whereupon the mixture exothermed to 75° C. The reaction was heated at 75° C. for 15 minutes, determined to be complete by LC/MS, poured into water (800 mL) and the formed brown precipitate filtered. The solid was dissolved in ethyl acetate, sodium sulfate and activated carbon was added and the mixture heated to 60° C. for 5 minutes. The mixture was filtered through Celite and the solvent removed under reduced pressure to give 13a as an off-white solid. Yield: 4.2 g, 70%.

TABLE 2
Azaindoles via Pathway B

| | |
|---|---|
| | 13 |
| 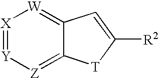 | |

| | W | X | Y | Z | T | R² |
|---|---|---|---|---|---|---|
| 13a | CH | CH | CH | N | NH | CH₂C₆H₅ |
| 13b | CH | CBr | CH | N | NH | CH₂SO₂C₆H₅ |
| 13c | CH | CF | CH | N | NH | CH₂CO₂Me |
| 13d | CH | CF | CF | N | NMe | CH₂NO₂ |
| 13e | CF | CF | CF | N | N-c-C₃H₅ | CH₂CN |
| 13f | C—Me | CF | CF | N | NMe | Me |
| 13g | CH | C—CN | C—CN | N | N—Me | CH₂SiMe₃ |
| 13h | CH | C—CO₂Me | C—CO₂Me | N | NH | CH₂PO(OMe)₂ |
| 13i | C—CF₃ | C—Br | C—Me | N | NH | CH₂PO(OMe)₂ |
| 13j | N | CH | CH | CH | NH | Me |
| 13k | N | C—Me | CH | CH | NH | CH₂COMe |
| 13m | N | C—Me | C—Me | N | NH | CH₃ |
| 13n | N | C—CO₂Me | C—Me | N | N—c-C₄H₇ | CH₂CN |
| 13o | CH | N | CH | N | N—c-C₃H₅ | CH₂NO₂ |
| 13p | N | CH | CH₂CO₂Me | CH | N—Me | Me |
| 13q | N | CH | C—2-thienyl | C—Me | N—Me | Me |
| 13r | CH | N | C—Me | N | O | Me |
| 13s | C—Me | N | C—Me | N | O | CH₂C₆H₅ |
| 13t | CH | C—CH₂NMe₂ | CH | N | O | CH₂SMe |
| 13u | N | C—c-C₃H₅ | N | CH | O | CH₂-p-FC₆H₄ |
| 13v | N | CH | CH₂F | N | O | CH₂C₆H₅ |
| 13w | CH | CH | N | N | S | Me |
| 13x | CH | CH | C—CH₂NMe₂ | N | S | CH₂C₆H₅ |
| 13y | C—c-C₃H₅ | N | C—NMe₂ | N | S | CH(Me)CO₂Me |
| 13z | CH | CH | C—OMe | N | S | CHMe₂ |

### 5-Bromo-2-((phenylsulfonyl)methyl)-1H-pyrrolo[2,3-b]pyridine (13b)

This was similarly prepared as for 13a, except 5-bromo-3-(3-(phenylsulfonyl)-prop-1-ynyl)pyridin-2-amine (12b) was used. Intermediate 8b was prepared from 5-bromo-3-iodopyridin-2-amine (9a).

### Methyl 2-(5-fluoro-1H-pyrrolo[2,3-b]pyridine-2-yl)acetate (13c)

This was similarly prepared as for 13a, except methyl 4-(2-amino-5-fluoropyridin-3-yl)but-3-ynoate (12c) was used. Intermediate 8c was prepared from 5-fluoro-3-iodopyridin-2-amine (9b).

### 5,6-Difluoro-1-methyl-2-(nitromethyl)-1H-pyrrolo[2,3-b]pyridine (13d)

This was similarly prepared as for 13a, except 5,6-difluoro-N-methyl-3-(nitroprop-1-ynyl)pyridin-2-amine (12d) was used. Intermediate 12d was prepared from 5,6-difluoro-3-iodo-N-methylpyridin-2-amine (11d).

### 5,6-Difluoro-4-methyl-1H-pyrrolo[2,3-b]pyridine (13e)

This was similarly prepared as for 13a, except 4-(2-cyclopropylamino)-4,5,6-trifluoropyridin-3-yl)but-3-ynenitrile (12e) was used. Intermediate 12e was prepared from N-cyclopropyl-4,5,6-trifluoro-3-iodopyridin-2-amine (11e).

### 5,6-Difluoro-1,2,4-trimethyl-1H-pyrrolo[2,3-b]pyridine (13f)

This was similarly prepared as for 13a, except 5,6-difluoro-N,4-dimethyl-3-(prop-1-ynyl)pyridine-2-amine (12f) was used. Intermediate 12f was prepared from 5,6-difluoro-3-iodo-N,4-dimethylpyridin-2-amine (11f).

### 5,6-Dicyano-1-methyl-2-(trimethylsilyl)methyl)-1H-pyrrolo[2,3-b]pyridine (13g)

This was similarly prepared as for 13a, except 5,6-dicyano-N-methyl-3-(3-(trimethylsilyl)prop-1-ynyl)pyridine-2-amine (12g) was used. Intermediate 12g was prepared from 5,6-dicyano-3-iodo-N-methylpyridin-2-amine (11g).

### 5-Bromo-4-(trifluoromethyl)-6-methyl-1H-pyrrolo[2,3-b]pyridine (13h)

This was similarly prepared as for 13a, except dimethyl 3-(5,6-di(methoxy-carbonyl)-2-aminopyridin-3-yl)prop-2-ynylphosphonate (1 2h) was used. Intermediate 12h was prepared from dimethyl 6-amino-5-iodopyridine-2,3-dicarboxylate (9g).

### Dimethyl (5-bromo-4-(trifluoromethyl)-6-methyl-1H-pyrrolo[2,3-b]pyridine-2-yl)methylphosphonate (13i)

This was similarly prepared as for 13a, except dimethyl 3-(2-amino-5-bromo-4-(trifluoromethyl)-6-methylpyridin-3-yl)prop-2-ynylphosphonate (12i) was used. Intermediate 12i was prepared from 5-bromo4-(trifluoromethyl)-3-iodo-6-methylpyridin-2-amine (9h).

### 2-Methyl-1H-pyrrolo[3,2-b]pyridine (13j)

This was similarly prepared as for 13a, except 2-(2-(prop-1-ynyl)pyridin-3-amine (12j) was used. Intermediate 12j was prepared from 2-iodopyridin-3-amine (9i).

### 2,3-Dimethyl-5H-pyrrolo[2,3-b]pyrazine (13k)

This was similarly prepared as for 13a, except 5-(3-amino-6-methylpyridin-2-yl)-pent-4-yn-2-one (12k) was used. Intermediate 12k was prepared from 2-iodo-6-methylpyridin-3-amine (9j).

### 2,3,6-Trimethyl-5H-pyrrolo[3,2-b]pyrazine (13m)

This was similarly prepared as for 13a, except 5,6-dimethyl-3-(prop-1-ynyl)-pyrazin-2-amine (12m) was used. Intermediate 8m was prepared from 3-iodo-5,6-dimethylpyrazin-2-amine (9k).

### Methyl 6-(cyanomethyl)-5-cyclobutyl-3-methyl-5H-pyrrolo[3,2-b]pyrazine-2-carboxylate (13n)

This was similarly prepared as for 13a, except methyl 6-(3-cyanoprop-1-ynyl)-5-(cyclobutylamino)-3-methylpyrazine-2-carboxylate (12n) was used. Intermediate 12n was prepared from methyl 5-(cyclobutylamino)-6-iodo-3-methylpyrazine-2-carboxylate (11n).

### 7-Cyclopropyl-6-(nitromethyl)-7H-pyrrolo[2,3-d]pyrimidine (13o)

This was similarly prepared as for 13a, except N-cyclopropyl-5-(3-nitroprop-1-ynyl)pyrimidin-4-amine (120) was used. Intermediate 120 was prepared from N-cyclopropyl-5-iodopyrimidin-4-amine (110).

### Methyl 2-(1,2-dimethyl-1H-pyrrolo[3,2-b]pyridine-6-yl)acetate (13p)

This was similarly prepared as for 13a, except methyl 2-(5-(methylamino)-6-(prop-1-ynyl)pyridine-3-yl)acetate (12p) was used. Intermediate 12p was prepared from 5-iodo-2,6-dimethylpyrimidin-4-amine (11p).

### 1,2,7-Trimethyl-6-(thiophen-2-yl)-1H-pyrrolo[3,2-b]pyridine (13q)

This was similarly prepared as for 13a, except N,4-dimethyl-2-(prop-1-ynyl)-5-(thiophen-2-yl)pyridine-3-amine (12q) was used. Intermediate 12q was prepared from 2-iodo-N,4-dimethyl-5-(thiophen-2-yl)pyridin-3-amine (11q).

### 2,6-Dimethylfuro[2,3-d]pyrimidine (13r)

This was similarly prepared as for 13a, except 2-methyl-5-(prop-1-ynyl)pyrimidin-4-ol (12r) was used. Intermediate 12r was prepared from 5-iodo-2-methyl-pyrimidin-4-ol (11r).

### 6-Benzyl-2,4-dimethylfuro[2,3-d]pyrimidine (13s)

This was similarly prepared as for 13a, except 2,6-dimethyl-5-(phenylprop-1-ynyl)pyrimidin-4-ol (12s) was used. Intermediate 12s was prepared from 2,6-dimethyl-5-iodo-pyrimidin-4-ol (11s).

### N,N-Dimethyl(2-((methylthio)methyl)furo[2,3-b]pyridine-5-yl)methanamine (13t)

This was similarly prepared as for 13a, except 5-(N,N-dimethylaminomethyl)-3-(3-(methylthio)prop-1-ynyl)pyridine-2-ol (12t) was used. Intermediate 12t was prepared from 5-(N,N-dimethylaminomethyl)-3-iodopyridin-2-ol (11t).

### 6-(4-Fluorobenzyl)-2-cyclopropylfuro[3,2-d]pyrimidine (13u)

This was similarly prepared as for 13a, except 2-cyclopropyl-4-(3-(4-fluorophenyl)prop-1-ynyl)pyrimidin-5-ol (12u) was used. Intermediate 12u was prepared from 2-cyclopropyl-4-iodopyrimidin-5-ol (11u).

### 6-Benzyl-3-(fluoromethyl)furo[3,2-b]pyrazine (13v)

This was similarly prepared as for 1 3a, except 6-(fluoromethyl)-3-(3-phenylprop-1-ynyl)pyrazin-2-ol (12v) was used. Intermediate 12v was prepared from 6-(fluoromethyl)-3-bromopyrazin-2-ol (11v).

### 6-Methylthieno[2,3-c]pyridazine (13w)

This was similarly prepared as for 13a, except 4-(prop-1-ynyl)pyridazine-3-thiol (12w) was used. Intermediate 12w was prepared from 4-iodopyridazin-3-thiol (11w).

### (2-Benzylthieno[2,3-b]pyridine-6-yl)-N,N-dimethylmethanamine (13x)

This was similarly prepared as for 13a, except 6-((dimethylamino)methyl)-3-(3-phenylprop-1-ynyl)pyridine-2-thiol (12x) was used. Intermediate 12x was prepared from 6-((dimethylamino)methyl)-3-iodopyridine-2-thiol (11x).

### Methyl 2-(4-cyclopropyl-2-(dimethylamino)methyl)thieno[2,3-d]pyrimidin-6-yl)-propanoate (13y)

This was similarly prepared as for 13a, except methyl 4-(4-cyclopropyl-2-((dimethylamino)methyl)-6-mercaptopyrimidin-5-yl )-2-methylbut-3-ynoate (12y) was used. Intermediate 12y was prepared from 6-cyclopropyl-2-((dimethyl-amino)methyl)-5-iodopyrimidine-4-thiol (11y).

### 6-Methoxy-1H-pyrrolo[2,3-b]pyridine (13z)

This was similarly prepared as for 13a, except 6-methoxy-3-(3-methylbut-1-ynyl)pyridine-2-thiol (12z) was used. 12z was prepared from 3-iodo-6-methoxypyridine-2-thiol (11z).

### COMPARATIVE EXAMPLE

Unlike that disclosed in the prior art, the present invention allows a wide range of sensitive functional group substituents to be tolerated, i.e., remain unaffected by the reactions described herein. The present Compartive Example illustrates the benefits of the present invention over that disclosed by U.S. Pat. No. 6,384,235 to Henkleman.

For example, when compound 1 of Scheme 1 (where D is Br) is treated under previously reported conditions, no selectivity substitution of the pyridinyl halogens is observed. The Sonogashira reaction occurs indiscriminately at both of the halogenated positions. According to the methods of the present invention, the substitution reaction is limited to the iodide site, leaving the bromine site untouched and ready for further elaboration.

The process according to the present invention is not only tolerant of functional groups on the heterocycle, but also on the terminal alkyne. Materials have been successfully isolated where R2 is a sensitive functional moiety such as an alcohol, halogen and thiol while still maintaining selectivity (i.e., Sonogashira reaction at only one site) and eliminating undesired side reactions of the functionalized alkyne such as intramolecular reactivity with the heterocyle, intermolecular reactivity, decomposition under the rigorous conditions outlined in previous work, etc. 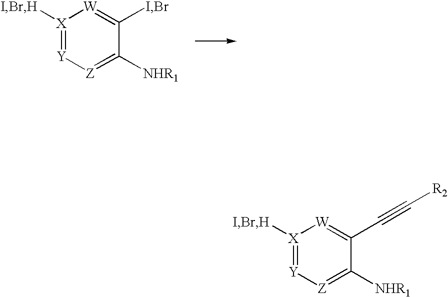
R₂=alcohol,alkyl, acetals, phosphonates, Halogens, amines, amides, thiols, etc. This type of tolerance will allow for the generation of high functionalized and multi-useful azaindole derivatives for the medicinal chemist.

Those skilled in the art will appreciate that numerous changes and modifications may be made to the preferred embodiments of the present invention and that such changes and modifications may be made without departing from the spirit of the invention. It is therefore intended that the appended claims cover all such equivalent variations as fall within the true spirit and scope of the invention.

What is claimed is:

1. A process for preparing a compound having the structure of Formula (I) 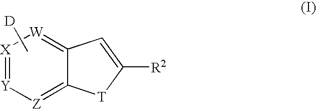
wherein
T is NR¹, oxygen, or sulfur, wherein
R¹ is hydrogen, substituted or unsubstituted C₁-C₆ alkyl, substituted or unsubstituted C₃-C₇ cycloalkyl, substituted or unsubstituted alkenyl, substituted or unsubstituted alkynyl, substituted or unsubstituted aralkyl, substituted or unsubstituted aryl, or substituted or unsubstituted heteroaryl;
R² is hydrogen, alkyl, haloalkyl, cycloalkyl, (CH₂)ₚOH, (CH₂)_{q}NR¹¹R¹², substituted or unsubstituted aryl, substituted or unsubstituted aralkyl, substituted or unsubstituted heteroaryl, fused substituted or unsubstituted aryl, fused substituted or unsubstituted heteroaryl, or CH(R³)J, wherein
J is hydrogen, alkyl, haloalkyl, CF₃, cycloalkyl, halogen, CHO, CH═NOH, CO₂H, CO₂-alkyl, CN, NO₂, PO(O-alkyl)₂, SO₂-alkyl, S-alkyl, SCF₃, SO₂-aryl, S-aryl, substituted or unsubstituted aryl, substituted or unsubstituted aralkyl, substituted or unsubstituted heteroaryl, fused substituted or unsubstituted aryl, fused substituted or unsubstituted heteroaryl;
R³ is selected from the group of hydrogen, alkyl, haloalkyl, CF₃, cycloalkyl, halogen, CHO, CH═NOH, CO₂H, CO₂-alkyl, CN, NO₂, PO(O-alkyl)₂, SO₂-alkyl, S-alkyl, SCF₃, SO₂-aryl, S-aryl, substituted or unsubstituted aryl, substituted or unsubstituted aralkyl, substituted or unsubstituted heteroaryl, fused substituted or unsubstituted aryl, fused substituted or unsubstituted heteroaryl;
R¹¹ and R¹² are independently hydrogen, alkyl, or alkanoyl;
p is 1 to 3;
q is 0 to 2;
W is CH, CR⁴, or N;
X is CH, CR⁵, or N;
Y is CH, CR⁶, or N;
Z is CH, CR⁷, or N, wherein the total number of nitrogens in W+X+Y+Z is 0-3, and optionally W+X, X+Y, or Y+Z could be joined as either a 5-7 member ring;
R⁴, R⁵, R⁶ and R⁷ are each independently hydrogen, haloalkyl, alkyl, cycloalkyl, (CH₂)ₚOH, halogen, CHO, CH═NOH, CO₂H, CO₂-alkyl, S-alkyl, SO₂-alkyl, S-aryl, (CH₂)_{q}NR¹³R¹⁴, alkoxy, CF₃, SCF₃, NO₂, SO₃H, OH, substituted or unsubstituted aryl, substituted or unsubstituted aralkyl, substituted or unsubstituted heteroaryl, fused substituted or unsubstituted aryl, fused substituted or unsubstituted heteroaryl;
R¹³ and R¹⁴ are independently hydrogen, alkyl, or alkanoyl; and D is H or Br,
the method comprising the steps of:
(a) reacting a compound of the formula 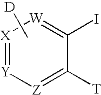
with an acetylene compound selected from the group consisting of 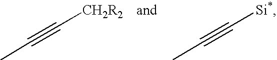
wherein R₂, D, T, W, X, Y, and Z are as previously defined, I is an iodine atom, and
Si* is a silyl-containing acetylene protecting group; and
(b) cyclizing the product of step (a) in a protic solvent.

2. The method of claim 1 wherein T is oxygen.

3. The method of claim 1 wherein T is sulfur.

4. The method of claim 1 wherein T is NR¹.

5. The method of claim 4 wherein R¹ is H.

6. The method of claim 4 wherein R² is CH(R³)J wherein R², R³, and J are as previously defined.

7. The method of claim 6 wherein at least one of W, X, Y, or Z is CR⁴, CR⁵, CR⁶, or CR⁷, respectively.

8. The method of claim 1 wherein at least one of W, X, Y, or Z is CR⁴, CR⁵, CR⁶, or CR⁷, respectively.

9. The method of claim 1 wherein the compound in step (a) is reacted with

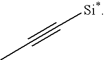

10. The method of claim 9 wherein the silyl-containing acetylene protecting group is selected from the group consisting of trimethylsilyl (TMS), diethylsilyl, tri-isopropylsilyl (TriPS), triethylsilyl, dimethylphenylsilyl, and t-butyl dimethylsilyl (TBDMS).

11. The method of claim 10 wherein the silyl-containing acetylene protecting group is a trimethylsilyl group.

12. The method of claim 1 wherein the compound in step (a) is reacted with

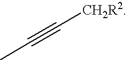

13. The method of claim 12 wherein R² is CH(R³)J wherein R², R³, and J are as previously defined.

14. The method of claim 1 wherein Z is N; X is CR⁵.

15. The method of claim 14 wherein R⁵ is Br.

16. The method of claim 14 wherein R² is benzyl.

17. The method of claim 1 wherein step (a) is performed in toluene.

18. The method of claim 17 wherein step (a) is performed at no greater than 45° C.

19. The method of claim 1 wherein step (a) is performed in toluene.

20. The method of claim 1 wherein the protic solvent is selected from the group consisting of n-butanol, tert-butanol, iso-butanol, iso-propanol, propanol, ethanol, methanol, and mixtures thereof.

21. The method of claim 20 wherein the protic solvent is butanol.

22. The method of claim 22 wherein D is Br and further comprising the step of substituting the Br.

23. A compound having the structure of Formula (I) 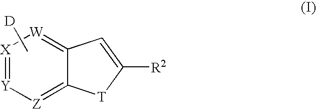
wherein T is NR¹ wherein R¹ is substituted or unsubstituted C₁-C₆ alkyl, substituted or unsubstituted C₃-C₇ cycloalkyl, substituted or unsubstituted alkenyl, substituted or unsubstituted alkynyl, substituted or unsubstituted aralkyl, substituted or unsubstituted aryl, or substituted or unsubstituted heteroaryl;
R² is hydrogen, alkyl, haloalkyl, cycloalkyl, (CH₂)ₚOH, (CH₂)_{q}NR¹¹R¹², substituted or unsubstituted aryl, substituted or unsubstituted aralkyl, substituted or unsubstituted heteroaryl, fused substituted or unsubstituted aryl, fused substituted or unsubstituted heteroaryl, or CH(R³)J, wherein
J is hydrogen, alkyl, haloalkyl, CF₃, cycloalkyl, halogen, CHO, CH═NOH, CO₂H, CO₂-alkyl, CN, NO₂, PO(O-alkyl)₂, SO₂-alkyl, S-alkyl, SCF₃, SO₂-aryl, S-aryl, substituted or unsubstituted aryl, substituted or unsubstituted aralkyl, substituted or unsubstituted heteroaryl, fused substituted or unsubstituted aryl, fused substituted or unsubstituted heteroaryl;
R³ is selected from the group of hydrogen, alkyl, haloalkyl, CF₃, cycloalkyl, halogen, CHO, CH═NOH, CO₂H, CO₂-alkyl, CN, NO₂, PO(O-alkyl)₂, SO₂-alkyl, S-alkyl, SCF₃, SO₂-aryl, S-aryl, substituted or unsubstituted aryl, substituted or unsubstituted aralkyl, substituted or unsubstituted heteroaryl, fused substituted or unsubstituted aryl, fused substituted or unsubstituted heteroaryl;
R¹¹ and R¹² are independently hydrogen, alkyl, or alkanoyl;
p is 1 to 3;
q is 0 to 2;
W is CH, CR⁴, or N;
X is CH, CR⁵, or N;
Y is CH, CR⁶, or N;
Z is CH, CR⁷, or N, wherein the total number of nitrogens in W+X+Y+Z is 0-3, and optionally W+X, X+Y, or Y+Z could be joined as either a 5-7 member ring;
R⁴, R⁵, R⁶ and R⁷ are each independently hydrogen, haloalkyl, alkyl, cycloalkyl, (CH₂)ₚOH, halogen, CHO, CH═NOH, CO₂H, CO₂-alkyl, S-alkyl, SO₂-alkyl, S-aryl, (CH₂)_{q}NR¹³R¹⁴, alkoxy, CF₃, SCF₃, NO₂, SO₃H, OH, substituted or unsubstituted aryl, substituted or unsubstituted aralkyl, substituted or unsubstituted heteroaryl, fused substituted or unsubstituted aryl, fused substituted or unsubstituted heteroaryl;
D is H or Br; and
R¹³ and R¹⁴ are independently hydrogen, alkyl, or alkanoyl.

24. The compound of claim 23 wherein R¹ is H.

25. The compound of claim 23 wherein R² is CH(R³)J wherein R², R³, and J are as previously defined.

26. The compound of claim 25 wherein at least one of W, X, Y, or Z is CR⁴, CR⁵, CR⁶, or CR⁷, respectively.

27. The compound of claim 23 wherein at least one of W, X, Y, or Z is CR⁴, CR⁵, CR⁶, or CR⁷, respectively.

28. The compound of claim 23 wherein R² is CH(R³)J wherein R², R³, and J are as previously defined.

29. The compound of claim 23 wherein Z is N; X is CR⁵.

30. The compound of claim 29 wherein R⁵ is Br.

31. The compound of claim 30 wherein R² is benzyl.

32. A compound having the structure of Formula (I) 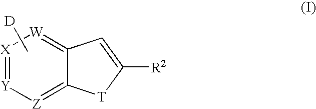
wherein T is selected from NR¹, oxygen, sulfur, wherein
R¹ is hydrogen, substituted or unsubstituted C₁-C₆ alkyl, substituted or unsubstituted C₃-C₇ cycloalkyl, substituted or unsubstituted alkenyl, substituted or unsubstituted alkynyl, substituted or unsubstituted aralkyl, substituted or unsubstituted aryl, or substituted or unsubstituted heteroaryl;
R² is CH(R³)J, wherein J is hydrogen, alkyl, haloalkyl, CF₃, cycloalkyl, halogen, CHO, CH═NOH, CO₂H, CO₂-alkyl, CN, NO₂, PO(O-alkyl)₂, SO₂-alkyl, S-alkyl, SCF₃, SO₂-aryl, S-aryl, substituted or unsubstituted aryl, substituted or unsubstituted aralkyl, substituted or unsubstituted heteroaryl, fused substituted or unsubstituted aryl, fused substituted or unsubstituted heteroaryl;
R³ is selected from the group of hydrogen, alkyl, haloalkyl, CF₃, cycloalkyl, halogen, CHO, CH═NOH, CO₂H, CO₂-alkyl, CN, NO₂, PO(O-alkyl)₂, SO₂-alkyl, S-alkyl, SCF₃, SO₂-aryl, S-aryl, substituted or unsubstituted aryl, substituted or unsubstituted aralkyl, substituted or unsubstituted heteroaryl, fused substituted or unsubstituted aryl, fused substituted or unsubstituted heteroaryl;
p is 1 to 3;
q is 0 to 2;
W is CH, CR⁴, or N;
X is CH, CR⁵, or N;
Y is CH, CR⁶, or N;
Z is CH, CR⁷, or N, wherein the total number of nitrogens in W+X+Y+Z is 0-3, and optionally W+X, X+Y, or Y+Z could be joined as either a 5-7 member ring;
R⁴, R⁵, R⁶ and R⁷ are each independently hydrogen, haloalkyl, alkyl, cycloalkyl, (CH₂)ₚOH, halogen, CHO, CH═NOH, CO₂H, CO₂-alkyl, S-alkyl, SO₂-alkyl, S-aryl, (CH₂)_{q}NR¹³R¹⁴, alkoxy, CF₃, SCF₃, NO₂, SO₃H, OH, substituted or unsubstituted aryl, substituted or unsubstituted aralkyl, substituted or unsubstituted heteroaryl, fused substituted or unsubstituted aryl, fused substituted or unsubstituted heteroaryl;
D is H or Br; and
R¹³ and R¹⁴ are independently hydrogen, alkyl, or alkanoyl.

33. The compound of claim 32 wherein T is oxygen.

34. The compound of claim 32 wherein T is sulfur.

35. The compound of claim 32 wherein T is NR¹.

36. The compound of claim 35 wherein R¹ is H.

37. The compound of claim 32 wherein R² is CH(R³)J wherein R², R³, and J are as previously defined.

38. The compound of claim 37 wherein at least one of W, X, Y, or Z is CR⁴, CR⁵, CR⁶, or CR⁷, respectively.

39. The compound of claim 32 wherein at least one of W, X, Y, or Z is CR⁴, CR⁵, CR⁶, or CR⁷, respectively.

40. The compound of claim 32 wherein R² is CH(R³)J wherein R², R³, and J are as previously defined.

41. The compound of claim 32 wherein Z is N; X is CR⁵.

42. The compound of claim 41 wherein R⁵ is Br.

43. The compound of claim 41 wherein R² is benzyl.
